(19) United States
(12) Patent Application Publication
(10) Pub. No.: US 2015/0190509 A1
(43) Pub. Date: Jul. 9, 2015

(54) RESINIFERATOXIN SOLUTION

(71) Applicant: Thomas Giller, Wintersingen (CH)

(72) Inventor: Thomas Giller, Wintersingen (CH)

(73) Assignee: MESTEX AG, Zürich (CH)

(21) Appl. No.: 14/419,297

(22) PCT Filed: Aug. 3, 2012
(86) PCT No.: PCT/CH2012/000179

§ 371 Date: Feb. 3, 2015

(51) Int. Cl. A61K 47/02; A61K 47/10; B65D 85/00; A61K 47/14; A61K 47/06; A61K 45/06; A61K 31/357; A61K 47/20

(52) U.S. Cl. CPC A61K 47/02; A61K 31/357; A61K 47/10; A61K 47/20; A61K 47/14; A61K 47/06; A61K 45/06; B65D 85/70

(57) ABSTRACT

The invention relates to a resiniferatoxin solution having an enhanced storage stability, in which the resiniferatoxin is dissolved in a body-compatible solvent which contains a protective gas in solution, wherein the amount of the protective gas is at least 1 wt % of the saturation amount of the protective gas in the solvent at ambient temperature and normal pressure. The resiniferatoxin dissolved in the resiniferatoxin solution remains stable for a relatively long period and exhibits a reduced tendency to cling to polymeric surfaces.

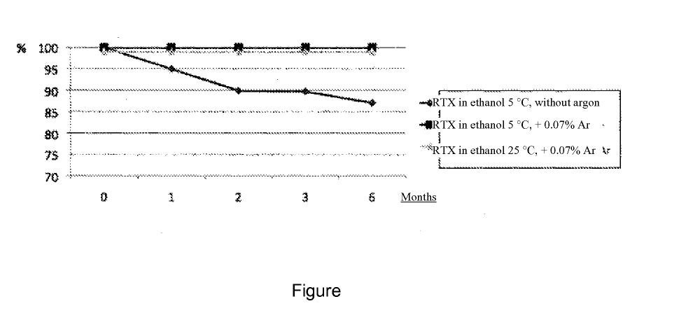

The invention relates to a resiniferatoxin solution according to the preamble of Claim 1.

Resiniferatoxin (RTX) can be used for a wide variety of purposes, primarily in medicine and in medical or pharmacological research. It originates from the plant Euphorbia resinifera and it has the desired property of being active at the TRPV1 receptor, and presumably at other sites, already at the smallest dosages. It is thus biologically related to capsaicin and chemically it is a diterpene from the family of the daphnane structures. The molecule also has similarities with the phorbol ester family, but without any detectable tumor-suppressive action. However, one difficulty in handling RTX is that it is usually used in quantities that are not visible to the naked eye and it is, in particular, very difficult to detect in combination with other substances. On the one hand, this is due to the extremely low required dosage which itself makes the detection difficult, and, on the other hand, RTX has the tendency of easily binding to a wide variety of surfaces and substances. An additional property of RTX is that, in the form of a powder or in a dissolved state including deep frozen (−20 ° C.), it has only limited stability and reacts with great susceptibility to oxidation by atmospheric oxygen or hydrolysis. The molecular weight is 628.7 g/mol, and the empirical formula is C₃₇H₄₀O₉. The storage accordingly occurs usually in the form of a powder, at −20 ° C. In the non-frozen state, it remains unstable and decomposes easily to ROPA (resiniferanol-9,13,14-orthopentyl acetate) and other fragments. For these reasons, RTX is thawed immediately before use and dissolved at ambient temperature with a solvent, typically ethanol or dimethyl sulfoxide (DMSO), in order to be then used further in this form.

This procedure is complicated and very imprecise because of the very small dosages or else it is very expensive.

Here, the aim of the invention is to provide a remedy. The problem of the invention is to provide a resiniferatoxin solution in which the dissolved resiniferatoxin:

(a) remains stable for a longer period; and

(b) has a reduced tendency to cling to polymeric surfaces.

The invention solves the posed problem with a resiniferatoxin solution that has the features according to claim 1.

The saturation amount for each protective gas that can be considered in the respective selected solvent (at ambient temperature and normal pressure) can be obtained easily from the appropriate handbooks or can also be determined by a simple measurement. One liter of alcohol, for example, dissolves 120 mL nitrogen gas at 19° C. and normal pressure (Rompps Chemie-Lexikon, 8th edition, 1979, p. 3984). The saturation amount for argon was determined to be 0.07 wt % at ambient temperature and normal pressure.

Surprisingly, it has been found that the resiniferatoxin when dissolved in the solvent enriched with protective gas remains very stable and can be stored even at ambient temperature (temperature range up to +25° C.) for several months (see the figure). Interestingly and unexpectedly, it is not sufficient to store frozen RTX under a protective gas, for example, argon, and then dissolve it with ethanol. Under these circumstances a loss of the RTX dissolved in ethanol occurs, on the order of magnitude of 10% in 3 months. However, if the RTX is dissolved together with argon in ethanol or in other body-compatible solvents, the loss at ambient temperature remains very low. As described in further detail below, RTX in the resiniferatoxin solution according to the invention even loses its undesired tendency to cling to polymeric surfaces.

The compositions indicated as examples below relate to highly concentrated supply solutions (stock solutions) of RTX, which are diluted appropriately before use (for example, with hyaluronic acid, water or mixtures thereof) and optionally mixed with buffer solutions, so that they can be injected into the patient.

### EXAMPLE 1

| Substance | Range in wt % |
|---|---|
| RTX | 0.000375 |
| Ethyl acetate | 0.00049 |
| Heptane | 0.00131 |
| Methylcyclohexane | 0.00001 |
| Ethyl alcohol | 99.92819 |
| Argon | 0.07 |
| Total | 100.000000% |

### EXAMPLE 2

| Substance | Range in wt % |
|---|---|
| RTX | 0.0006 |
| Ethyl acetate | 1.0 |
| Heptane | 3.0 |
| Methylcyclohexane | 0.04 |
| Dimethyl sulfoxide | 95.844 |
| Nitrogen | 0.11 |
| Total | 100.0000% |

### EXAMPLE 3

| Substance | Range in wt % |
|---|---|
| RTX | 0.001 |
| Heptane | 3.0 |
| Methylcyclohexane | 0.05 |
| Ethyl alcohol | 96.749 |
| Carbon dioxide | 0.2 |
| Total | 100.000% |

Surprisingly it is already sufficient to use a mixture of RTX, ethanol and dissolved argon alone; in the process, an RTX concentration of 3.75, 12.00 and 60.00 μg/mL has been established. As alternative to ethanol, dimethyl sulfoxide (DMSO) or another suitable body-compatible solvent or mixtures thereof can be selected.

As an alternative to argon, other inert gases or gas mixtures can also be used, for example: nitrogen N₂, CO₂, helium, neon, krypton, xenon or mixtures thereof.

It has been found to be particularly helpful for the protective gas to be introduced at increased pressure into a capsule (glass transparent or brown or colored otherwise; metal coated or uncoated) with the RTX supply solution or directly into the RTX supply solution.

In the process, the concentration of the protective gas measured in the liquid increases in comparison to its content at normal pressure. However, in principle, the gas can be introduced into the RTX supply solution using any of the procedures familiar to the person skilled in the art.

Use of the Supply Solution:

The resiniferatoxin solution according to the invention, after appropriate dilution (for example, with hyaluronic acid, water or mixtures thereof), is used to produce injectable individual doses or multiple doses. They contain an amount of RTX from 1 ng to 10 microgram, preferably 50 ng to 2 microgram, produced from the resiniferatoxin solution (stock solution) according to the invention with a concentration from 1 μg/mL to 50 μg/mL. However, the resiniferatoxin solution according to the invention can also be used for producing mixtures for external treatment, for example, ointments or creams, or for solutions that are used only temporarily (for example, bladder or mucosal membranes). The preferred uses relate to painful sites on the locomotor apparatus, in particular the intra-articular use in synovial joints. Other preferred sites are the synovial spaces and the area around tendons, the use in the nucleus pulposus, in and around the intervertebral disk, on ligaments and their attachment sites as well as in and around joint capsules, as well as the use in and around painful teeth. Other uses relate to the nervous system, for example, intra- or periganglionic as well as intra-, epi- and peridural and intra-arachnoid uses on the vertebral column and on the cerebrum. The uses cover humans and animals.

An additional positive property of the resiniferatoxin solution according to the invention is the fact that the substance is stabilized even in a diluted, ready-for-use solution (i.e. diluted with a buffer solution and hyaluronic acid), so that it can also be used in combination with commercial, polymer plastic syringes which are usually produced from polypropylene, polyethylene, polyurethane and other materials. To date, it has been considered absolutely necessary for the RTX solution not to come in contact with the polymer, because it binds immediately to it. However, in the manufacturing form represented here, it can be drawn into a polypropylene syringe, kept there for 10 minutes, and then injected into the patient without measurable loss of RTX.

The advantages achieved by the invention thus are essentially that the resiniferatoxin solution according to the invention has, on the one hand, a substantially higher stability and thus storage capacity, and, on the other hand, a reduced tendency to cling to polymeric surfaces.

The single figure shows a graph on which the time in months is plotted on the x axis and the stability of the dissolved RTX is plotted on the y axis. Here, three measurement series are represented: the first (diamond measurement points) shows the results for RTX dissolved in ethanol at 5° C. without additional protective gas (argon), the second (square measurement points) shows the results for RTX dissolved in ethanol at 5° C. with a content of 0.07 wt% argon, and the third (triangle measurement points) shows the results for RTX dissolved in ethanol at 25° C. and with a content of 0.07 wt % argon.

The first measurement series for the unstabilized RTX solution shows a continuous decrease of the RTX content. The second and third measurement series demonstrate that even at a relatively high temperature of 25° C. the RTX content remains at 100% without change for months.

Further advantageous embodiments of the invention can be commented on as follows:

In a particular embodiment, the amount of the protective gas is at least 2 wt %, preferably at least 4 wt % of the saturation amount of the protective gas in the solvent at ambient temperature and normal pressure. The amount of the protective gas can advantageously be at least 10 wt %, preferably at least 30 wt % of the saturation amount of the protective gas in the solvent at ambient temperature and normal pressure.

In a further embodiment, the solvent comprises ethanol or dimethyl sulfoxide or mixtures thereof. In addition, the solution can also contain one or more of the following solvents:

a) heptane, preferably at most 1.0 wt %;

b) ethyl acetate, preferably at most 0.4 wt %;

c) butanol

d) acetone,

e) tert-butyl methyl ether, preferably at most 0.2 wt %;

f) dichloromethane, preferably at most 0.2 wt %;

g) methylcyclohexane, preferably at most 0.01 wt %.

In a particular embodiment, the solution contains 0.00010-3.0000 wt %, preferably 0.000375-0.01000 wt % resiniferatoxin.

In an additional embodiment, the solution contains 1 to 60 μg/mL, preferably 3 to 50 μg/mL resiniferatoxin.

The protective gas can be selected from the following group: argon, nitrogen, carbon dioxide, helium, neon, krypton, xenon or mixtures thereof.

In a particular embodiment, the solution contains at most 0.03 wt %, preferably at most 0.01 wt % oxygen.

Advantageously, the resiniferatoxin solution has a pH from 7.0 to 9.0, preferably from 7.3 to 8.2.

In a particular embodiment, the resiniferatoxin solution contains, in addition, one or more of the following substances: sodium chloride, calcium chloride or potassium chloride. Advantageously, the quantitative ratio between RTX and protective gas M_{RTX}/M_{protective gas} is in the range from 0.04 to 10.0, preferably from 0.1 to 2.0.

In a particular embodiment, the resiniferatoxin solution comprises, in addition, a tricyclic antidepressant.

The resiniferatoxin solution according to the invention can be used in particular for treating intra-articular pains.

In a particular embodiment, the resiniferatoxin solution is filled into a container which contains the protective gas. At least the inner wall of the container can be made of a polymeric material. The polymeric material can be selected from the following group: polypropylene, polyethylene, polyurethane or combinations thereof. Advantageously, the container can be formed as a syringe.

A method for producing the resiniferatoxin solution according to the invention consists in that the resiniferatoxin is dissolved in the solvent, and subsequently the solution obtained is enriched with the protective gas.

Although there are different embodiments of the present invention as described above, it must be understood in the sense that the different features can be used either alone as well as in any desired combination. This invention is therefore is not limited simply to the above-mentioned, particularly preferred embodiments.

1. A resiniferatoxin solution having an enhanced storage stability, wherein the resiniferatoxin is dissolved in a body-compatible solvent which contains an amount of a protective gas in solution, wherein the amount of the protective gas is at least 1 wt % of a saturation amount of the protective gas in the solvent at 25° C. and 1 atmosphere pressure.

2. The resiniferatoxin solution according to claim 1, wherein the amount of the protective gas is at least 2 wt %, of the saturation amount of the protective gas in the solvent at 25° C. and 1 atmosphere pressure.

3. The resiniferatoxin solution according to claim 2, wherein the amount of the protective gas is at least 10 wt % of the saturation amount of the protective gas in the solvent at 25° C. and 1 atmosphere pressure.

4. The resiniferatoxin solution according to claim 1, wherein the solvent comprises ethanol or dimethyl sulfoxide or a mixture thereof.

5. The resiniferatoxin solution according to claim 1, wherein the solution further comprises one or more solvents selected from the group consisting of:
a) heptane;
b) ethyl acetate;
c) butanol;
d) acetone;
e) tert-butyl methyl ether;
f) dichloromethane; and
g) methylcyclohexane.

6. The resiniferatoxin solution according to claim 1, wherein the solution contains 0.00010-3.0000 wt % resiniferatoxin.

7. The resiniferatoxin solution according to claim 1, wherein the solution contains 1 to 60 μg/mL resiniferatoxin.

8. The resiniferatoxin solution according to claim 1, wherein the protective gas is selected from the group consisting of: argon, nitrogen, carbon dioxide, helium, neon, krypton, xenon and mixtures thereof.

9. The resiniferatoxin solution according to claim 1, wherein the solution contains at most 0.03 wt %.

10. The resiniferatoxin solution according to claim 1, wherein the solution has a pH from 7.0 to 9.0.

11. The resiniferatoxin solution according to claim 1, wherein the solution further comprises sodium chloride, calcium chloride, potassium chloride or mixtures thereof.

12. The resiniferatoxin solution according to claim 1, the wherein a quantitative ratio between RTX and protective gas (M_{RTX}/M_{protective gas}) is in a range from 0.04 to 10.0.

13. The resiniferatoxin solution according to claim 1, further comprising a tricyclic antidepressant.

14. A method for the treatment of intra-articular pains in a patient, comprising injecting a resiniferatoxin solution according to claim 1 into the patient intra-articularly.

15. The resiniferatoxin solution according to claim 1, it wherein the solution is filled into a container which contains the protective gas.

16. The resiniferatoxin solution according to claim 15, wherein at least an inner wall of the container is made of a polymeric material.

17. The resiniferatoxin solution according to claim 16, wherein the polymeric material is selected from the group consisting of: polypropylene, polyethylene, polyurethane and combinations thereof.

18. The resiniferatoxin solution according to claim 15, wherein the container is in the form of a syringe.

19. A method for producing a resiniferatoxin solution according to claim 1, wherein the resiniferatoxin is dissolved in the solvent, and subsequently the solution obtained is enriched with the protective gas.
